(19) United States
(12) Patent Application Publication
(10) Pub. No.: US 2003/0041512 A1
(43) Pub. Date: Mar. 6, 2003

(54) Staggered walkers forest management process

(76) Inventor: Leland F. Hazeltine, Woodland, CA (US)

Correspondence Address: Mark C. Jacobs, Esq., 3033 El Camino Avenue, Sacramento, CA, 95821, US

(21) Appl. No.: 09/945,177

(22) Filed: Sep. 4, 2001

(51) Int. Cl. A01B 79/02; A01H 3/00; A01C 1/00

(52) U.S. Cl. 47/58.1 R

(57) ABSTRACT

A process for the eradication of undesirable plants on a plot of ground upon which young desired species are planted is disclosed. The desired trees or seedlings are covered with a sleeve that is preferably retained in place, followed by a parade of workers entering the field spraying herbicide from a wand, preferably held at waist level, spraying in a waterfall pattern, wherein each of the workers are preferably spread apart forwardly and laterally a finite distance in a staggered formation.

### FIELD OF THE INVENTION

This application relates to selective herbicidal treatment of defined plots of land by walking personnel.

### BACKGROUND OF THE INVENTION

In order to commercially propagate soft wood forests such as of pine, spruce, cedar and fir, it is necessary to eradicate the brush, weeds, grasses and other tree varieties, all of which are classified collectively as undesirables, because they compete for the water and sunlight of the forest. But the trick is to eradicate the undesirables without damaging the “desired species” usually soft woods of tree farms (cedar, pine, fir). In doing the eradication of the undesirables, worker safety must be considered, as well as effective use of herbicide to keep the cost of eradication to a minimum, while achieving a high percentage of success.

Accordingly, the process of this invention achieves the desired eradication with a high degree of success while providing for worker safety and protection of the “desired species”.

It is accordingly a first object to provide a new mode of protecting trees during the eradication of undesirables.

It is a second object to provide a technique for the application of herbicide to protected species of young trees.

It is a third object to provide an ecologically sound mode of protecting the desired species.

It is a fourth object to provide a mode of eradication of undesirables that takes into account worker safety.

These and other objects of the invention will in part be obvious and will in part appear hereinafter.

The invention accordingly comprises the device possessing the features properties and the relation of components which are exemplified in the following detailed disclosure and the scope of the application of which will be indicated in the appended claims.

For a fuller understanding of the nature and objects of the invention reference should be made to the following detailed description, taken in conjunction with the accompanying drawings.

This process can be carried out in several modes by workers marching through a field. The preferred mode encompasses a cadre of men walking in a spaced relationship to each other, staggered apart in a forwardly direction, spraying herbicide preferably from a waist high position from side to side, and preferably in a waterfall pattern, in a forest wherein the young desired trees have been paper sleeve covered and the sleeves optionally retained in place, such that the undesired species of plants are killed off, with little or no loss of the covered crop of desired specie(s). While paper sleeves that are breathable are the preferred sleeve material, a breathable plastic that will permit air to pass therethrough, but not the larger molecules of herbicide, may also be employed. Paper ones are preferred, as they are more easily biodegradable.

### BRIEF DESCRIPTION OF THE FIGURES

FIG. 1 is typical soft wood young tree such as of pine or fir.

FIG. 2 a typical cylindrical paper sleeve employed in this invention.

FIG. 3 is the paper sleeve of FIG. 2 in the process of being lowered over the young tree of FIG. 1.

FIG. 4 is a cutaway view of the tree of FIG. 1 disposed in the sleeve of FIG. 2 and the sleeve being retained in place.

FIG. 5 is a typical herbicide applicator person using the spraying technique of this invention for forest management.

FIG. 6 is a top plan view showing a cadre of workers in procession spraying a plot of land having sleeved trees, covered therein according to this invention.

FIG. 7 is a top plan view illustrating the motion used during the herbicide application.

FIG. 8 is a diagrammatic view used to explain FIG. 7.

FIG. 9 is a point in time post the spraying operation.

FIG. 10 is a diagrammatic view illustrating the formation of a cylindrical sleeve from a flat sheet.

FIG. 11 is a perspective view of an inverted truncated cone sleeve with two severable built in flaps for soil covering for retention.

### SUMMARY OF THE INVENTION

A process for forest management that permits a high degree of eradication of undesirables, while protecting the desired species from the herbicide used for eradication and further ensuring worker safety during the application procedure. The process includes covering and if necessary retaining a breathable sleeve in position over a young tree, and spraying the plot of ground having the covered young trees in a particular manner by having a plurality of workers preferably walk in a staggered offset pattern and apply herbicide in a preferably side to side motion at a waist high level.

### DESCRIPTION OF THE PREFERRED EMBODIMENT

In FIG. 1 there is shown a typical young seedling tree age one to three years. Typical species to be propagated include many of the soft commercial woods, spruce, pine, fir and cedar. The generic tree 35 may be any of these other desired species.

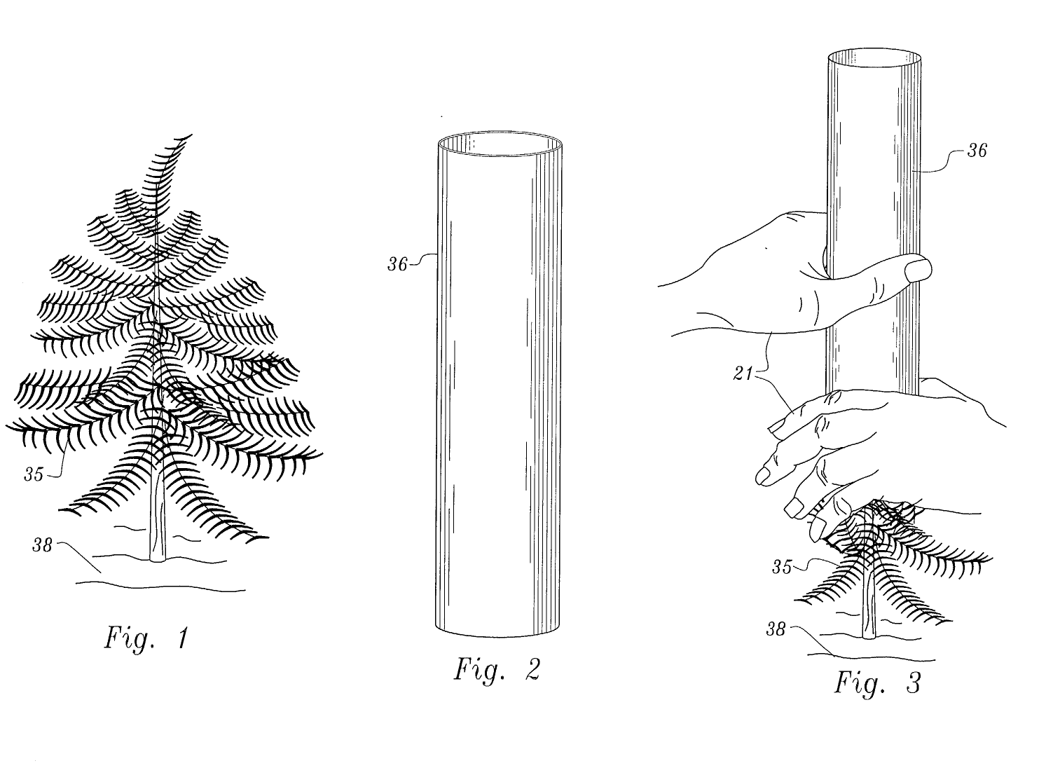

In FIG. 2 a typical paper sleeve within the confines of this invention is shown and is designated 36. The sleeve 36 is used to cover the tree 35 to prevent herbicide from contacting the desired species. Sleeve 36 is made by various manufacturers in combinations of cross sections and elongations that allows for a firm fit according to the tree at hand, such that a “grip” of the tree transpires. In some instances, it may be necessary to cover the base of the sleeve slightly, to prevent the sleeve from being blown off the tree, but in most instances such is not required. Most commercial sleeves have one of the two main panels slightly larger than the other such that the larger panel can be rolled over the smaller one, and formed into a cylinder. The sleeve is formed of sufficient length such that top, —the area above the height of the tree, may be folded over to “seal” the tree body against any entry of chemical herbicides into the sleeve. It is this slight extra length of paper that forms flap 37.

In FIG. 3 sleeve 36 is being lowered into position over tree 35 by the hands 21 of a worker. After the sleeve covers the tree as in FIG. 4, the worker may fold over a flap 37 to close off the sleeve at the top. He or she may optionally place a few pebbles 41 or some soil 40 around the base (bottom end) of the sleeve to retain the sleeve in position over the young tree, to thus keep the sleeve from blowing up or off. Pebbles or loose dirt may usually be found within an area of approximately one to two feet in any direction on the earth floor 38 suitable for this purpose.

FIG. 4, is a cutaway view of a sleeve to show the tree in its protected environment, protected that is from herbicide spray. Note the presence of soil 40 disposed around the base of the sleeve 36.

FIG. 10 illustrates the steps necessary to convert a flat sheet 60 with self adhesive section 61 on an edge thereof into a cylinder 36, by overlapping the adhesive edge onto the opposite edge of the sheet, by about one inch to form a top flap.

As noted earlier, the sleeves to be used may be cylindrical or inverted truncated cones. The latter is illustrated in FIG. 11. Here sleeve 136 is seen to have two built I scoring areas, 137, and a pair of vertical pre-scored lines 138 to render the separation of the two bottom wings easier. One score line 138 is not visible in this view. Crease lines 139 are for the bending of the two wings 137 outwardly such that they can be covered with dirt as may be desired. These bottom wings, which spread apart are to be mentally distinguished from the top flaps which form an overlap. It is within the skill of the art to have truncated cone sleeves with both bottom wings and a top flap as well.

The process aspect of this invention employs a very specific spraying technique. Thus, reference is made to FIG. 5. Here worker 20, one of many employed for the project carries a backpack sprayer 30 which has tubing 31 in fluid communication with a spray wand 32 which employs a wand with a trigger or valve thereon, which wand has a particular nozzle 47 thereon, and which is known to provide a fan like dispersal, which when carried out at waist-high elevation results in a waterfall effect or arc type of spray. Such nozzles are available in the marketplace from several sources, including Spray Systems Inc. of Wheaton, Ill., among others. Sprayers from Spray Systems using 5500 series type “X” nozzle, permit the spray stream to be adjusted to achieve the desired drop size and pattern to be employed according to this invention. The worker 20 maintains his hand 21 having the spray wand 32 therein at about his or her own waist level. Arrows 34 shows this height to be about thirty-four to thirty-six inches above the ground, depending on the height of the worker for position and retention of the spray wand 32.

As can be seen the spray pattern 33 that emanates from the nozzle 47 is a waterfall shape pattern that falls in front of the feet of the worker 20.

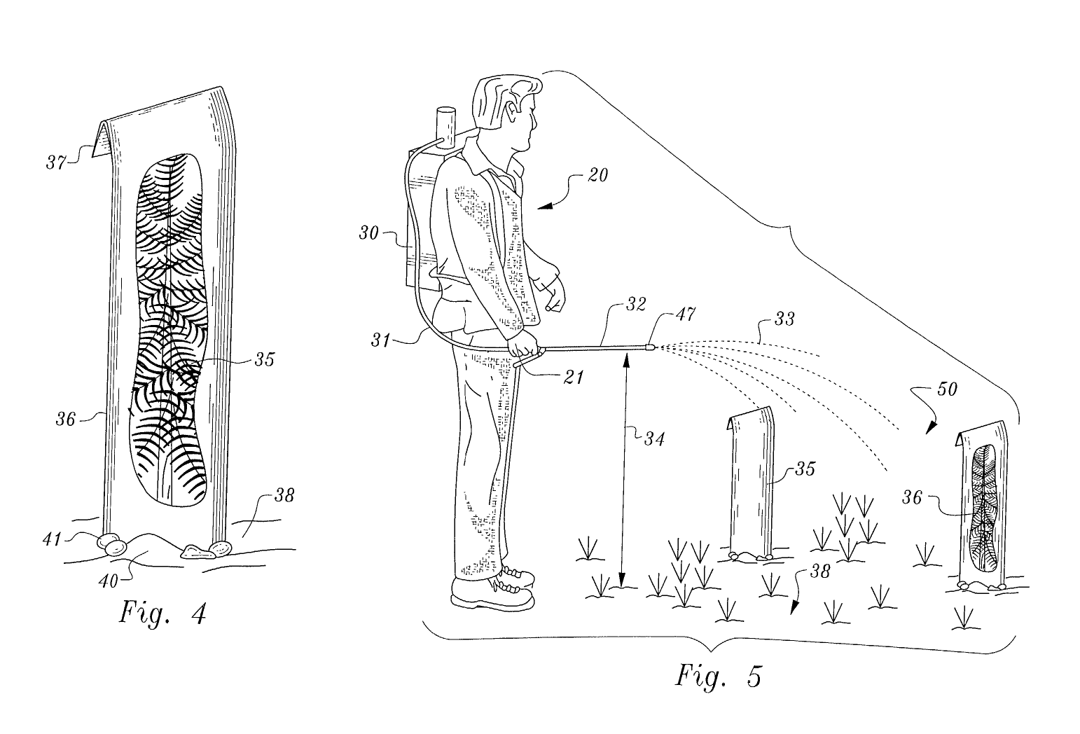

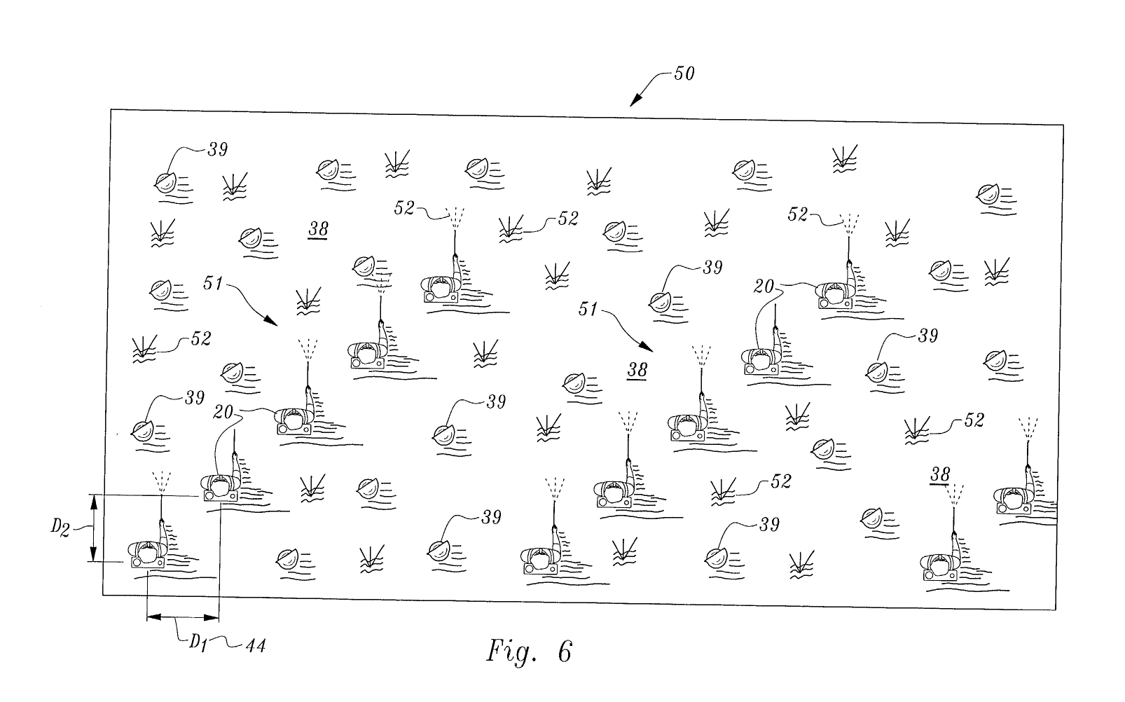

The workers 20, according to this invention's preferred mode, are to walk through the field 50 in a special pattern. That is, the workers 20 are preferably to walk in a staggered offset pattern, about ten feet apart as shown by D1, designator 44 and each worker is about ten feet in front of the worker to his or her left. This disposition of five workers is called work group 51. The work group may consist of five to ten workers, or if the plot is extra large the group may be up to twenty-five workers. Shown in FIG. 6 is a field of two work groups 51, and part of a third group to fill in at the border of the field.

In this top plan view the undesirables to be sprayed are shown as squiggly lines 52. These undesirables may include weeds, grasses, even wild flowers, as well as undesirable broad species such as oak and maple which are relatively slower growing trees. The covered trees 39 are shown by the square sleeve with the X therein. The covered trees may be present in a random pattern as shown or in a regular column and row pattern as may be desired.

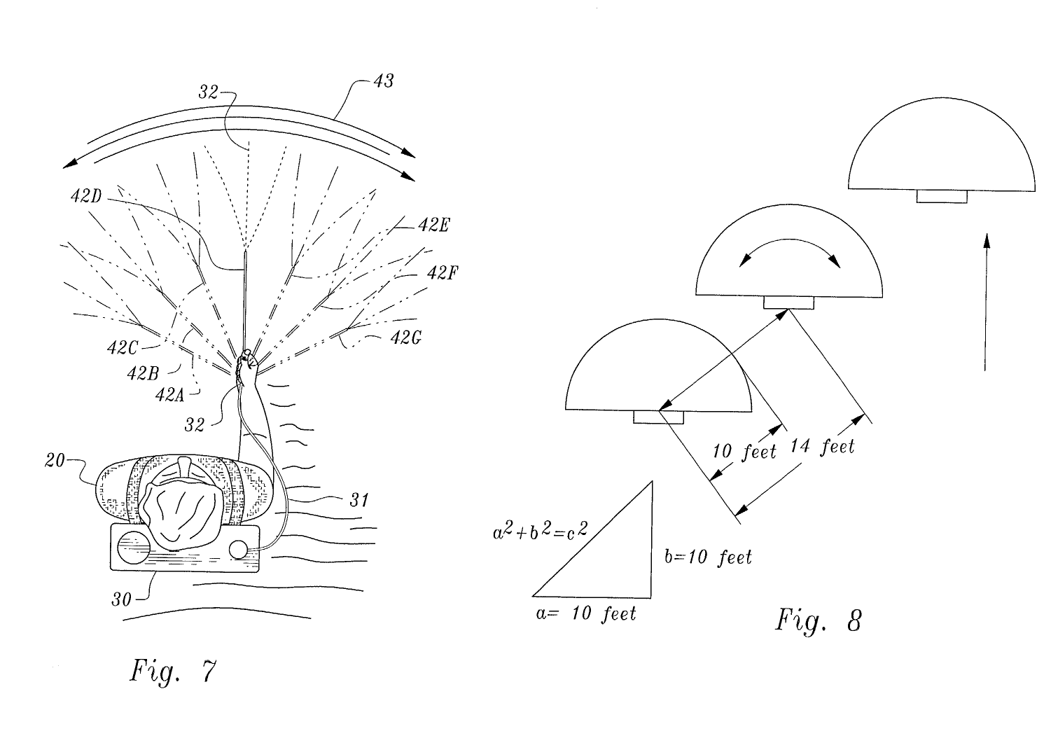

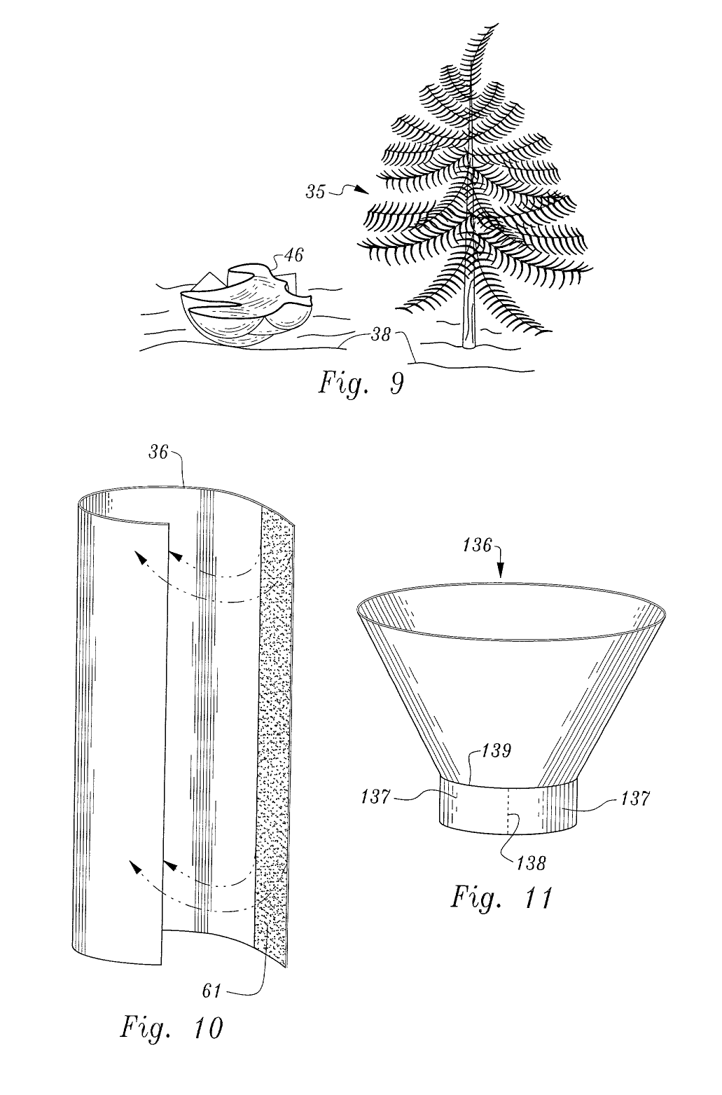

In FIG. 7 the application technique of each worker 20 of the work group 51 is depicted. As noted supra, each worker 20 carries a backpack 30 which has tubing 31 connected to a spray head 32. The spray pattern as noted as a waterfall pattern from top to bottom and a side to side motion as depicted in line 42 points 42A-G illustrates seconds in time for the first left to right sweep of the spray wand 32.

The diagram constituting FIG. 8 illustrates how the positioning of one worker relative to another worker's location permits all workers to spray in the field without spraying each other. Ten feet separation and ten foot staggered disposition, provides 14.1 ft. between workers. The placement relies on the application of the Pythagorean theorem in that the square root of c²=the sum of a²+b², yields 14.1 based on a=10 and b=10.

By spraying from the waist in a waterfall pattern, worker safety is ensured as spray is aimed directly ahead and not at the applicator's face, eyes and body. The movement of the hands of the worker from a side to side motion insures total coverage on each side of the worker's body. Yet by the adjacent worker being ten feet ahead and behind, the possibility of spraying a coworker is virtually eliminated.

Adjustment of the spray distance from the focal point of the worker is something well within the skill of the art, by a careful manipulation of the spray head trigger and by an artful choice of nozzle 47. Nozzle choice is also within the skill of the artisan.

When the cadre moves forward each worker sprays left to right, back to left, et cetera. Each worker can follow the edge of the spray pattern applied by the worker immediately ahead, thus creating an overlap effect in which each worker applies fifty percent of the total applied volume to a particular area. This insures that no area will be missed as could occur when each worker applies one-hundred percent of the volume needed to cover the travel swath. Better chemical deposition is also assured as the error range from skips and overlaps is fifty percent to one-hundred and fifty percent versus zero percent to two-hundred percent. If an unusual amount of undesirables is encountered by an individual worker at a specific location, he/she can give the area an extra dose of herbicide, and still not endanger fellow workers or upset the program as a whole. This ensures full coverage of the area to be treated.

By allowing each worker to apply fifty percent of the volume in a twenty-foot swath, such that one-hundred percent is applied by two persons to a particular area or zone, better worker protection is achieved, as the angle of release from the wand can be kept horizontal, whereas a wider swath would require an upward arcing of the spray stream, thereby possibly exposing the worker to a greater risk of contamination from the spray.

The use of the newly proposed twenty foot+/−swath in conjunction with the subsequent worker overlap staggered movement as recited in this invention, rather than the currently accepted and used forty to fifty foot swath width with spray stream being released at an upward angle to achieve the greater width, will provide increased worker safety, while achieving better and more uniform results. My desired results are obtained by having the resulting spray volume travel path from the nozzle of the wand to the target starting substantially parallel to the ground from an elevation at the worker's waist height. In such manner the possibility of small drops landing on adjacent workers is significantly reduced when so applied from waist height, versus the application when the sprayer is held high and aimed high.

While the discussion has centered around the preferred mode of applying herbicide by workers walking in a staggered pattern and spraying from waist-high positions of the wand, other walking worker modes can be employed, though with lesser results. One such mode is known as the directed broadcast mode of application. In this procedure, a cadre of workers walk in a single parallel line, much like the soldiers of war in the times of the U.S. Revolution or War between the States. Each worker carries a backpack of herbicide and applies spray in an arc toward the area to be treated and away from the sleeve protected tree variety. See my copending application, Ser. No.______filed______, Docket Number 1591 filed concurrently herewith.

When and as the spraying procedure has been completed, with the addition of a day or two in case of high wind the work groups will then remove the individual sleeves 36. The sleeves will be crumpled up as shown by designator 46 and left for collection or merely permitted to remain on the earth to decompose.

Since most herbicides have a half life, the efficacy of residual herbicide in the sleeves will be of little or no detriment to the environment. See FIG. 8.

It is seen that I have disclosed an improved process for eradicating undesirables while protecting the desired species from the impact of herbicide spray. The mode of application utilized herein assures total coverage of the land plot in question, while simultaneously providing for worker safety.

While the discussion also has centered on the removal of grasses, weeds and even hardwoods from commercial soft wood tree farms, the process of this invention can also be used to eradicate undesirables from hard wood farms or forests as well.

The process of this invention is particularly beneficial in areas struck by fire where hundreds of seedling are planted to reforest an area.

The sleeves utilizable herein may be of a paper or any biodegradable breathable material such as cotton or certain plastics. These materials will allow air in, but not the much larger molecules of the herbicides. The process can be used with seedlings and young trees of the desired species.

While I prefer to use adjacent soil crumbles as the retention medium, other biodegradable weights may be employed.

While I have suggested 10′×10′ separations of work group members, smaller distances can be employed by adjustment in the choice of nozzle to provide for a smaller waterfall dispensing of herbicide. Thus laterally and forwardly they could be a little as six feet apart and each would employ a three-foot radius of spray from these respective spray wands instead of a five-foot spray left and right. The workers of the group enter and exit the plot to be treated walking forwardly, in the staggered offset formation pattern.

As can be seen from the drawings the sleeves are cylindrical in configuration, and preferably tapered cylinders with the bottom being a bit wider than the top. Such sleeves 36 may also be referred to as truncated cones, with parallel ends. By selecting the preferred version, a snug fit of the sleeve according to the general anatomy of the conifers over which they are to be applied can be achieved. Thus, the choice of a cylinder or a tapered cylinder will depend on the variety of tree to be protected from spray impaction.

While the primary purpose of this invention is for the protection of conifers, forests of other trees, such as eucalyptus which is not a conifer, can be protected and treated according to this invention, using the tapered cone sleeve. Oaks and maples and other hardwood young trees, may best be protected if they are the desired crop by use of the truncated cone sleeves.

It has been determined that the sleeves, which are produced from overlapped stock, of either a rectangular or trapezoidal configuration, having a 1/2inch overlap to form the particular cone, can vary in size. The size to be used in any one particular job will vary according to the size of tree or seedling to be protected.

For cylindrical sleeves, the size can range from about a six-inch circumference with an elevation of about ten-inches, to a ten-inch circumference×20″, to a 12″ circumference×24″ tall. {Circumference=ΠD} Truncated cones are preferably formed in the configuration wherein the top opening has a circumference of 1Z and the bottom has a circumference of 2Z. A typical example of the sizing of a tapered cylinder would have Z=2, for a 6.28 inch circumference top with a 12.56 inch lower circumference, and a 12″ elevation. Both types of sleeves, cylindrical and truncated cone, can be produced with or without a flap, as is shown in the drawings.

Little or no discussion has been spent on the spray apparatuses to be employed, as they are deemed conventional. The backpack mounted sprayer connects to the tubing leading to a spray wand which has a trigger actuation means thereon. The spray tip on the wand is the basis for determining the pattern of spray. This is seen to be analogous to a multi functional garden hose nozzle which permits a stream, a mist or a fan shaped delivery pattern for the water. A fan shape at waist height, straight out will give rise to a waterfall pattern in both situations.

Since certain changes may be made in the above apparatus without departing from the scope of the invention herein involved, it is intended that all matter contained in the above description and shown in the accompanying drawings shall be interpreted as illustrative and not in a limiting sense.

I claim:

1. A process for the elimination of undesirables in favor of the encouragement of growth of selected desired species of trees which process comprises:
a. placing a sleeve over each desired specie seedling in a plot of land;
b. retaining the sleeve covering the desired specie member to prevent the sleeve from blowing away;
c. forming work groups of a finite number of member workers, wherein the workers are spaced apart in both the lateral and forwardly directions in a staggered formation; and
each worker spraying herbicide, in a waterfall pattern while walking in the staggered offset formation, while walking from the point of entry into the plot of ground to be treated to the point of exit of the plot of ground to be treated.

2. The process of claim 1 wherein the workers spray at waist level in a side to side motion.

3. The process of claim 1 plus the additional step of subsequently removing the sleeves from the desired species after the spraying is completed.

4. The process of claim 1 including forming a flap on each sleeve for the reception of weights to ensure the retention of the sleeve over the desired species.

5. The process of claim 3 wherein the sleeve to cover the desired species are retained in place by placing indigenous soil on the sleeve's flap.

6. The process of claim 1 wherein the workers of the work group are spaced ten feet laterally apart, and ten feet forwardly apart.

7. The process of claim 4 further including the steps of removal of the weights and crumbling of the sleeves and placement of the crumpled sleeves on the ground.

8. A process for eradicating undesirable tree species and protecting desirable tree species in a plot of ground from harm which comprises:
a. forming a flap on a paper sleeve and covering each member of the desired species, one sleeve per desirable tree;
b. placing a paper sleeve with the flap extending outwardly over each desired specie, with the flap resting on the ground;
c. retaining each sleeve in place by suitable weights as it is placed over the desired specie;
d. forming work groups of a finite number of member workers, spaced a specified distance apart, and a specified distance forward for each succeeding member worker of the group;
e. each worker applying herbicide in a waterfall pattern using a side to side motion, said workers spraying from the point of entry to the point of exist of the plot of ground to be treated; and
f. removing the sleeves from each desirable specie upon completion of the spraying.

9. The process of claim 8 further including crumpling each removed sleeve.

10. A process for the eradication of undesirable plants on a plot of ground upon which desired species are being propagated, which process comprises:
a. covering each of the desired species with a sleeve;
b. retaining the sleeve over the desired specie; and
c. spraying the plot of land containing the desired covered species with herbicide in a waterfall pattern by a moving group of workers.

11. The process of claim 10 wherein the workers enter and exit the plot being sprayed in a staggered order spaced apart.

12. The process of claim 11 wherein the workers are spaced 6-10 feet apart laterally.

13. The process of claim 11 wherein each worker of a work group is spaced 6-10 feet in front of the adjacent worker.

14. A process for the elimination of undesirables in favor of the encouragement of growth of selected desired species of trees which process comprises:
a. placing a sleeve over each desired specie seedling in a plot of land;
b. retaining the sleeve covering the desired specie member to prevent the sleeve from blowing away;
c. devising work groups of a finite number of member workers, wherein the workers are spaced apart in a linear formation; and
each worker spraying herbicide, while walking in formation from the point of entry into the plot of ground to be treated to the point of exit of the plot of ground to be treated.

15. The process of claim 14 wherein each worker sprays in a waterfall pattern.

16. The process of claim 15 wherein the workers spray by moving a spray wand at waist level in a side to side motion.

17. The process of claim 14 plus the additional step of subsequently removing the sleeves from the desired species after the spraying is completed.

18. The process of claim 14 including forming a flap on each sleeve for the reception of weights to ensure the retention of the sleeve over the desired species.

19. The process of claim 18 wherein the sleeve to cover the desired species are retained in place by placing indigenous soil on the sleeve's flap.

20. A process for the elimination of undesirables in favor of the encouragement of growth of selected desired species of trees which process comprises:
a. placing a sleeve over each desired specie seedling in a plot of land;
b. retaining the sleeve covering the desired specie member to prevent the sleeve from blowing away;
c. forming a flap on each sleeve for the reception of weight to ensure the retention of the sleeve over the desired species;
d. creating work groups of a finite number of member workers, wherein the workers are spaced apart in both the lateral and forwardly directions in a staggered formation;
each worker spraying herbicide at waist level in a side to side motion, in a waterfall pattern while walking in the staggered offset formation, from the point of entry into the plot of ground to be treated to the point of exit of the plot of ground to be treated and; and
e. the additional step of subsequently removing the sleeves from the desired species after the spraying is completed.

21. A sleeve for use in the protection of young trees during herbicidal spraying, which sleeve comprises:
a truncated cone having a first larger diameter and a second smaller diameter, and having a tubular section adjoining the smaller diameter, the tubular section including two pre-scored lines which when cut, permits the to sections of the former tubular section to be folded outwardly at the junction with the smaller diameter.

22. The sleeve of claim 21 wherein the elevation of the sleeve is less than about eight-inches tall.
